(19) United States
(12) Patent Application Publication
(10) Pub. No.: US 2026/0083684 A1
(43) Pub. Date: Mar. 26, 2026

(54) SKIN BARRIER

(71) Applicant: Michael T. Stanek, Fort Wayne, IN (US)

(72) Inventor: Michael T. Stanek, Fort Wayne, IN (US)

(21) Appl. No.: 19/338,654

(22) Filed: Sep. 24, 2025

Related U.S. Application Data
(60) Provisional application No. 63/698,774, filed on Sep. 25, 2024.

(51) Int. Cl. A61K 9/70

(52) U.S. Cl. CPC A61K 9/7084

(57) ABSTRACT

A skin barrier film system includes a film layer, an adhesive, an impregnated treatment and a removable layer. The film layer is shaped to accommodate a physical attribute of a patient. The adhesive is applied to at least part of the film layer. The impregnated treatment layer is coupled to the film layer. The removable layer is removably coupled to the adhesive, the film layer and/or the impregnated treatment layer.

### CROSS REFERENCE TO RELATED APPLICATIONS

This is a nonprovisional application of provisional application no. 63/698,774, entitled “SKIN BARRIER FILM”, filed Sep. 25, 2024, which is incorporated herein by reference.

### BACKGROUND OF THE INVENTION

### 1. Field of the Invention

The present invention relates a barrier system applied to a person's skin.

### 2. Description of the Related Art

Skin barrier films are specialized products used to protect the skin from damage caused by moisture, friction, adhesives, and bodily fluids. They are used in clinical and personal care settings. Skin barrier films are transparent coatings that may be breathable coatings applied to the skin to form a protective layer. They are typically applied by way of sprays, wipes, and foam applicators. The films that result from these application techniques are often made with silicone polymers like dimethicone, which are hypoallergenic and non-comedogenic.

Barrier films are used in Ostomy care for protecting peristomal skin from stoma output and adhesive trauma. They are used for wound care for shielding periwound skin from exudate and dressing adhesives. Further, they are used for incontinence management to prevent moisture-associated skin damage, such as incontinence-associated dermatitis.

Of course, the product is applied to clean, dry skin and the film is allowed to form with as the solvent evaporates, leaving a thin, flexible film. With the film acting as a barrier against irritants while allowing the skin to breathe and heal.

A problem with these sorts of films is that they require time to form, which is not particularly possible with infants. Another problem is that the solvents can be irritating to the skin.

What is needed in the art is a way of placing a medicated layer beneath a film with the film having anatomical accommodations.

### SUMMARY OF THE INVENTION

The invention relates to skin protection films.

The present invention provides a skin barrier film system that includes a film layer, an adhesive, an impregnated treatment and a removable layer. The film layer is shaped to accommodate a physical attribute of a patient. The adhesive is applied to at least part of the film layer. The impregnated treatment layer is coupled to the film layer. The removable layer is removably coupled to the adhesive, the film layer and/or the impregnated treatment layer.

The present invention in another embodiment provides a method of applying a skin barrier film to a patient, including the steps of: positioning, removing, aligning and contacting. The positioning step includes positioning the patient to receive at least one skin barrier film. The removing step includes the removing of a removable layer removably coupled to at least one of an adhesive, a film layer and an impregnated treatment layer. The aligning step is the alignment of the film layer that is shaped to accommodate a physical attribute of a patient with the physical attribute. The contacting step includes the contacting of the impregnated treatment layer that is coupled to the film layer with skin of the patient.

The present invention in another embodiment provides a skin barrier film system including a film layer being shaped to accommodate a physical attribute of a patient; an adhesive applied to a portion of a perimeter of the film layer; and a removable layer removably coupled to the adhesive on the film layer.

Advantageously the present invention is directed to anatomically shaped treatments with a film barrier to protect the skin against moisture, fecal matter and other irritants.

### BRIEF DESCRIPTION OF THE DRAWINGS

The above-mentioned and other features and advantages of this invention, and the manner of attaining them, will become more apparent and the invention will be better understood by reference to the following description of an embodiment of the invention taken in conjunction with the accompanying drawing, wherein:

FIG. 1 is an abstract view of an embodiment of a skin barrier system of the present invention;

FIG. 2 is a cross-sectional view of the skin barrier system of FIG. 1;

FIG. 3 is a view of an infant's bottom illustrating a medical condition in the form of a rash;

FIG. 4 is a view of a prior art film;

FIG. 5 is a plan view of an embodiment of the skin barrier system of the present invention having an anatomical shape;

FIG. 6 is another view of the skin barrier system of FIG. 5 having a removable layer removed;

FIG. 7 is yet another view of the skin barrier system of FIGS. 5 and 6 applied to a baby human;

FIG. 8 is still yet another view of the skin barrier system of FIGS. 5-7 applied to a baby human with the removal of another layer;

FIG. 9 is still yet another view of the skin barrier system of FIGS. 5-8 applied to a baby human with just the impregnated treatment and film layer applied to the baby;

FIG. 10 is a further view of the skin barrier system of FIGS. 5-9 applied to a baby human with the removal of the film layer leaving just the impregnated treatment area on the baby;

FIG. 11 is a view of another embodiment of the skin barrier system of the present invention applied to a person's neck;

FIG. 12 is a view of yet another embodiment of the skin barrier system of the present invention applied to the nipples of breasts;

FIG. 13 is a view of still yet another embodiment of the skin barrier system of the present invention applied to a heal of a foot; and

FIG. 14 is a view of a further embodiment of the skin barrier system of the present invention applied to the top of a foot.

Corresponding reference characters indicate corresponding parts throughout the several views. The exemplification set out herein illustrates at least one embodiment of the invention, and such exemplification is not to be construed as limiting the scope of the invention in any manner.

### DETAILED DESCRIPTION OF THE INVENTION

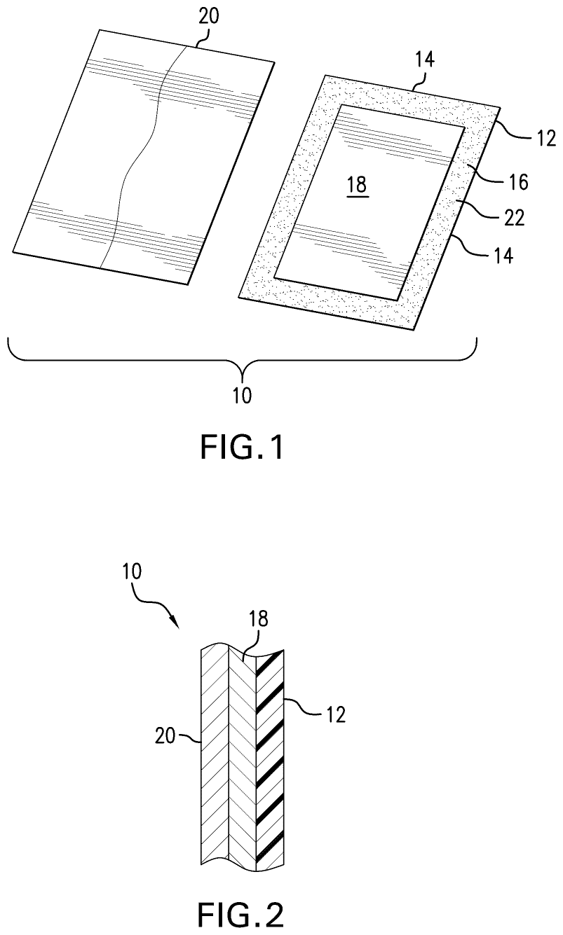

Referring now to the drawings, and more particularly to FIGS. 1 and 2, there is abstractly shown a skin barrier film system 10 having a film layer 12, edges 14, an adhesive 16, an impregnated treatment area 18, a removable layer 20, and an adhesive cover layer 22.

Among the embodiments of the Skin Barrier Film of the present invention a primary condition addressed is the condition of diaper dermatitis. Diaper dermatitis is an inflammatory skin condition of the diaper area, which affects millions of babies and toddlers each year. Symptoms consist of redness/irritation that can be uncomfortable and sometimes even painful to children. Prior art solutions include barrier creams, which are the treatment of choice. Barrier creams are messy, difficult to apply and do not form an actual barrier between urine/fecal matter and the affected skin.

Skin barrier film system 10 of the present invention is impregnated with soothing Aloe and/or Zinc oxide or other dermatitis treatment that is easily applied to a child's bottom. The anatomical pre-cut film forms an actual physical barrier to soiled diapers. This allows the child's skin to heal pain and irritation free.

Skin barrier film system 10 is applied by first, cleansing the child's bottom with a wet wipe or mild soapy cloth. Ensure that the cleansed area is clean and free from soil while allowing it to dry completely. Apply precut film 10 over the irritated diaper rash.

To remove skin barrier film system 10 simply peel film 12 off of the skin from edge 14 and discard. Replace film system 10 every 3-4 hours or if visibly soiled. Of course, if the rash does not improve or any additional concerning symptoms develop, seek medical attention immediately.

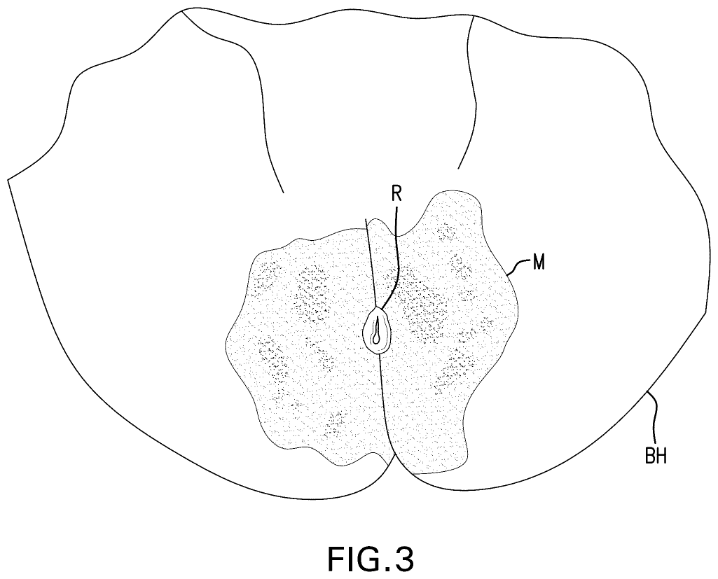

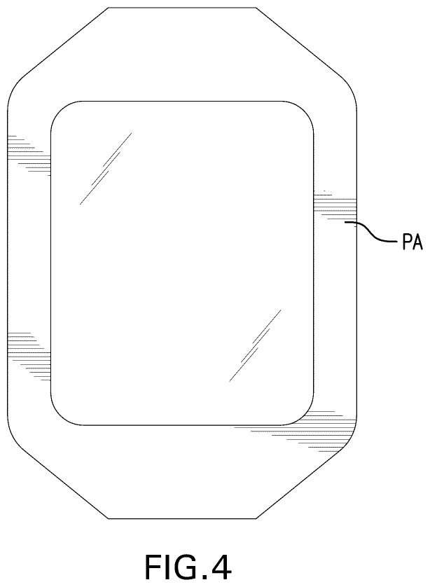

Now, additionally referring to FIG. 3 there is illustrated a baby human BH, having a medical condition M.

Now, additionally referring to FIG. 4 there is shown a prior art barrier PA that is often used to be applied over, for example, an IV site. Barrier PA has an adhesive edge area that adheres to the skin around the IV site and the middle part flexes over the IV to allow visual analysis of the site. There is no treatment associated with this prior art barrier. Barrier PA, is flat, has symmetrical straight perimeter lines and does not have a layer 18, nor does the prior art system have a perimeter with anatomical accommodating edges.

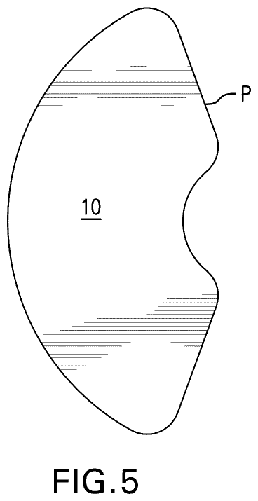

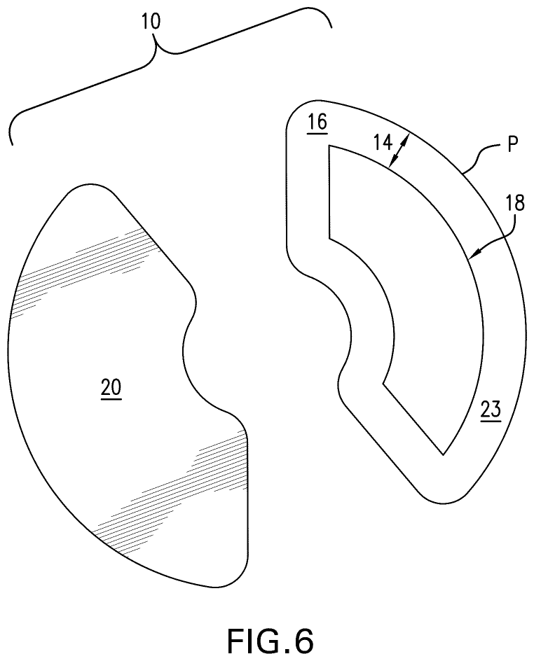

Now, additionally referring to FIGS. 5-10, there is illustrated some steps taken to use skin barrier film system 10. In FIG. 5 there is illustrated a bottom of baby human BH, with an anatomically shaped skin barrier film system 10, that is shaped to lie on one cheek of baby BH's bottom. An inner smaller arc is arranged relative to rectum R so that a majority of the skin affected by medical condition M is treated. While the figures illustrate the treatment of one side of baby BH, it is understood that the other side of baby BH is similarly treated.

System 10 is shown and should be considered to have not only a two dimensional flat shape, but also a three dimensional shape corresponding to the shape of a cheek of the rear of baby BH. In FIG. 6, removable layer 20 is removed from film 12, adhesive 16, impregnated treatment area 18, and adhesive cover layer 22.

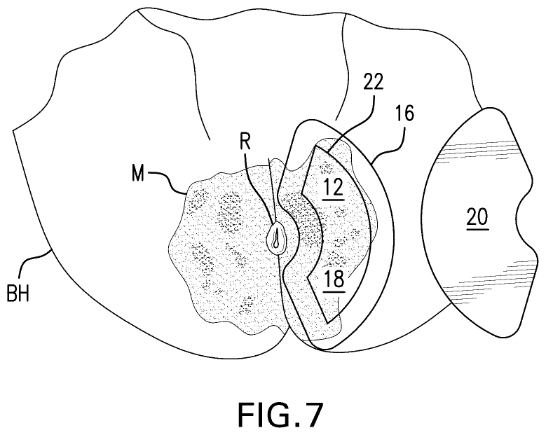

In FIG. 7, impregnated treatment area 18 is positioned and applied to an area experiencing a rash M, and adhesive cover layer 22 has not been removed. It is contemplated that adhesive 16, may only partially surround treatment area 18 as shown or may completely surround area 18, or be completely shared, so that adhesive 16 and treatment area 18 extend completely across the same areas.

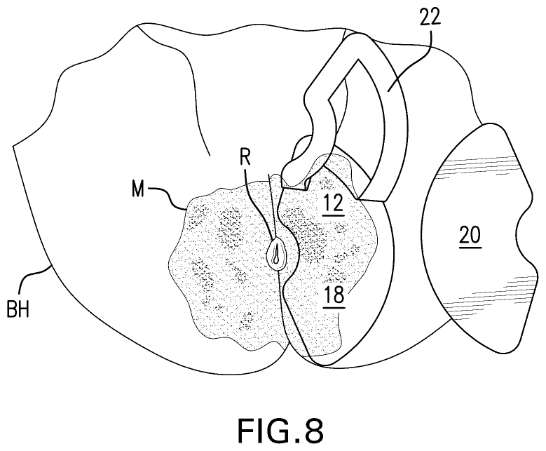

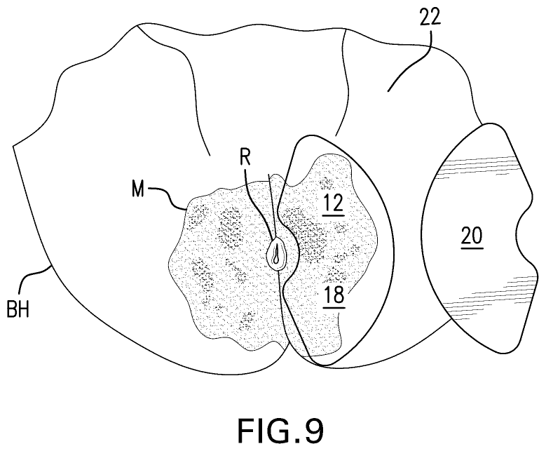

In FIG. 8, impregnated treatment area 18 remains on baby BH as adhesive cover layer 22 is removed from film 12, allowing an edge of film 12 to adhere to the skin. Once adhesive cover layer 22 is removed then film 12 with treatment 18 is on the skin, as shown in FIG. 9 and is worn to provide the needed treatment to the skin. This is the effective use of system 10, and is present in this condition until a removal or change is warranted.

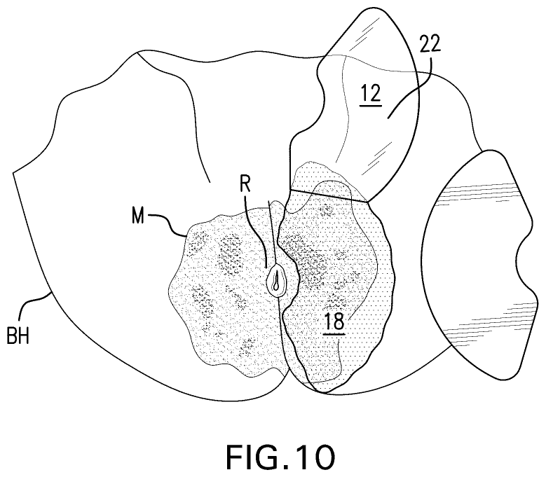

FIG. 10 illustrates the removal of film 12 from the skin and at least some of treatment 18 remains on the skin, with film 12 now being discarded.

It is contemplated that an inner surface of film 12, which contacts treatment layer 18, may have some physical characteristics that serve to retain treatment layer 18 when removable layer 20 is removed from the rest of system 10.

The present invention advantageously provides a physical barrier between a soiled diaper and the child's skin to accelerate healing of the skin being treated. The invention is easy to apply and remove, while providing a substantially uniform treatment to the affected area of skin. The anatomical shape of the invention allows it to readily conform to the skin and provide maximum protection.

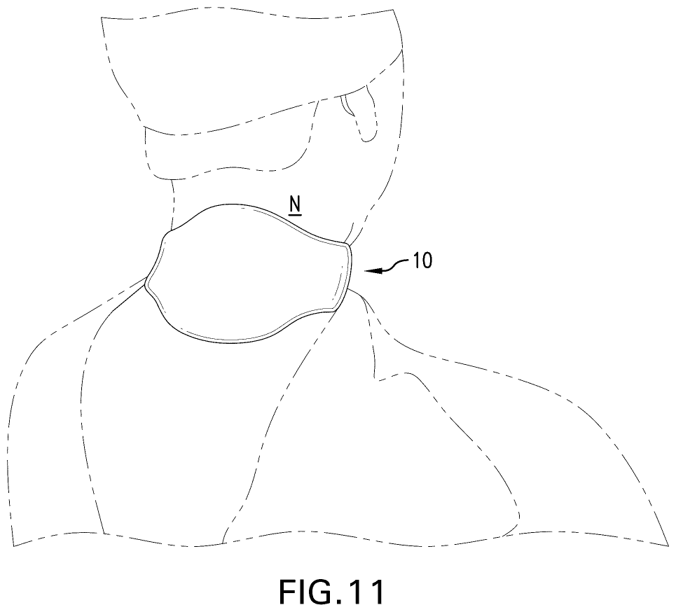

Now, additionally referring to FIG. 11 there is illustrated another embodiment of skin barrier system 10, with at least a portion of system 10 being shown applied to a neck N. In this embodiment system 10 takes on the anatomical shape of a neck and is used as a wetsuit neck protector-this can be placed on neck N prior to placing on a wetsuit. This advantageously can prevent neck chafing during swimming with a wetsuit.

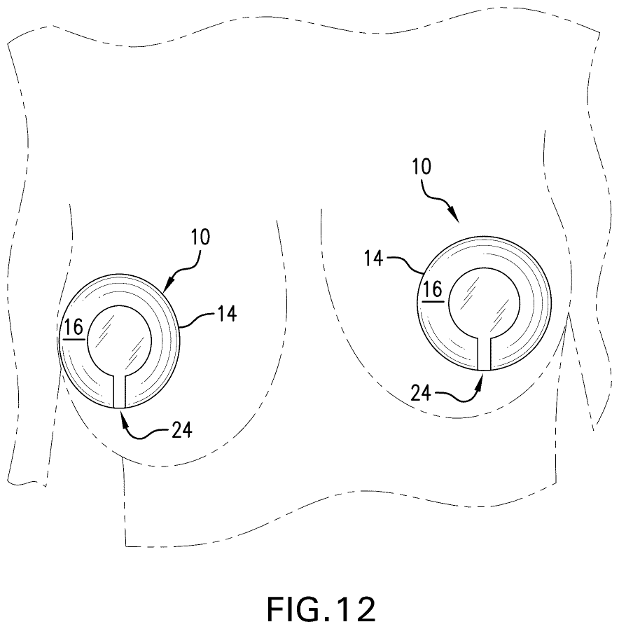

Now, additionally referring to FIG. 12 there is illustrated another embodiment of skin barrier system 10, with at least a portion of system 10 being shown applied over nipples. In this embodiment system 10 takes on the anatomical shape of a nipple with a raised, non-adhesive middle area. In this configuration there is an addition of an open channel 24. So that nipple barrier 10 acts as a nipple shield 10 with sweat channel 24 being part of the system to help protect sensitive areas during endurance sports, such as racing and training.

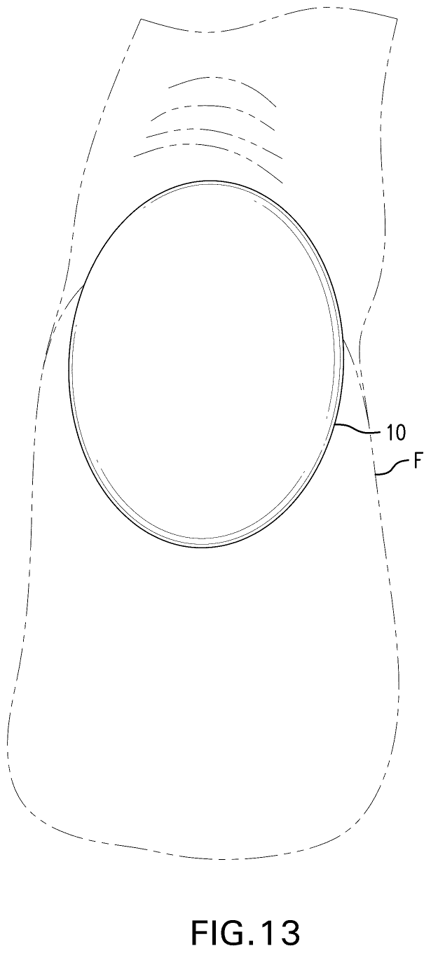

Now, additionally referring to FIG. 13 there is illustrated another embodiment of skin barrier system 10, with at least a portion of system 10 being shown applied to a foot F. In this embodiment system 10 takes on the anatomical shape of a heel.

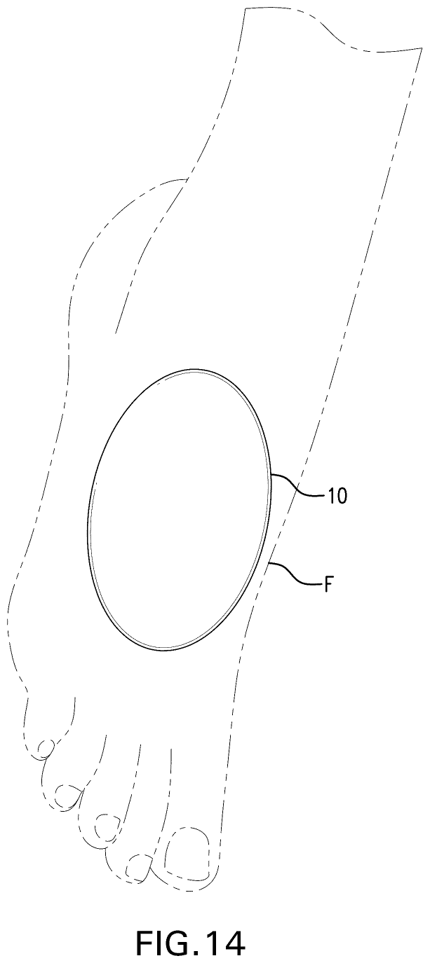

Now, additionally referring to FIG. 14 there is illustrated another embodiment of skin barrier system 10, with at least a portion of system 10 being shown applied to a top portion of foot F. In this embodiment system 10 takes on the anatomical shape of a top portion of foot F. In each case system provides ergonomically shaped patches can you placed over areas prone to blistering such as on the heel and fore foot, as shown in FIGS. 13 and 14, or on other parts of the body.

As discussed above, skin barrier film system 10 includes a film layer 12 being shaped to accommodate a physical attribute R of a patient to treat a medical condition M. An adhesive 16 is coupled to at least part of film layer 12. Impregnated treatment layer 18 is coupled to film layer 12, and removable layer 20 is removably coupled to at least one of the adhesive 16, the film layer 12 and the impregnated treatment layer 18. Removable layer protects the internal part of system 10, until it is removed and discarded when the balance of system 10 is applied to a patient.

Skin barrier film system 10 is arranged and shaped to accommodate the physical attribute R of the patient such as rectum R of patient BH. Film layer 12, in particular is shaped to allow positioning of the skin barrier film system adjacent to the rectum R with the impregnated treatment area 18 being in contact with the skin of patient BH.

Film layer 12, in a first embodiment, has a side with a concave shape that is positionable adjacent to rectum R, as can be seen in some of the figures. Film layer 12 also has a side opposite of the side with the concave shape which has a convex shape. The concave side and the convex side each have a center-point that is substantially the same in some applications.

Another way of viewing the shape of the first embodiment is that film layer 12 has an arcuate parentheses-type shape allowing the film layer to be positionable adjacent to both sides of rectum R by using two units of system 10. The parentheses-type look is like ( ), with rectum R located therebetween.

Film layer 12 of one skin barrier film system 10 is positionable on one cheek of the patient's bottom and another film layer 12 is positionable on another cheek of the patient's bottom.

Adhesive 16 and impregnated treatment layer 18 can overlap on film layer 12. Both adhesive 16 and treatment 18 are compatible and are not irritating to the skin. When film 12 is removed from the skin, impregnated treatment layer 18 substantially remains on the skin of the patient.

### COMPONENT REFERENCE LISTING

10. skin barrier film system
12. film layer
14. edges
16. adhesive
18. impregnated treatment area
20. removable layer
22. adhesive cover layer
24. Channel
BH. Baby human
F. Foot
M. Medical condition
N. Neck
P. Perimeter
PA. Prior art
R. Rectum

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A skin barrier film system, comprising:
a film layer being shaped to accommodate a physical attribute of a patient;
an adhesive applied to at least part of the film layer;
an impregnated treatment layer coupled to the film layer; and
a removable layer removably coupled to at least one of the adhesive, the film layer and the impregnated treatment layer.

2. The skin barrier film system of claim 1, wherein the physical attribute of the patient for which the film layer is shaped is a rectum of the patient, the film layer being shaped to allow positioning of the skin barrier film system adjacent to the rectum with the impregnated treatment area being in contact with the skin of the patient.

3. The skin barrier film system of claim 2, wherein the film layer has a side with a concave shape that is positionable adjacent to the rectum.

4. The skin barrier film system of claim 3, wherein the film layer has a side opposite of the side with the concave shape which has a convex shape.

5. The skin barrier film system of claim 4, wherein the concave side and the convex side each have a center-point that is substantially the same.

6. The skin barrier film system of claim 2, wherein the film layer has an arcuate parentheses-type shape allowing the film layer to be positionable adjacent to the rectum.

7. The skin barrier film system of claim 6, wherein the film layer of one skin barrier film system is positionable on one cheek of the patient's bottom and another film layer is positionable on another cheek of the patient's bottom.

8. The skin barrier system of claim 1, wherein the adhesive and the impregnated treatment layer overlap on the film layer.

9. The skin barrier system of claim 8, wherein the impregnated treatment layer substantially remains on the skin of the patient when the film layer is removed from the patient.

10. A method of applying a skin barrier film to a patient, comprising the steps of:
positioning the patient to receive at least one skin barrier film;
removing a removable layer removably coupled to at least one of an adhesive, a film layer and an impregnated treatment layer;
aligning the film layer that is shaped to accommodate a physical attribute of a patient with the physical attribute; and
contacting the impregnated treatment layer that is coupled to the film layer with skin of the patient.

11. The method of claim 10, wherein the physical attribute of the patient for which the film layer is shaped is a rectum of the patient, the film layer being shaped to allow positioning of the skin barrier film system adjacent to the rectum with the impregnated treatment area being in contact with the skin of the patient thereby avoiding the physical attribute.

12. The method of claim 11, wherein the film layer has a side with a concave shape that is positionable adjacent to the rectum.

13. The method of claim 12, wherein the film layer has a side opposite of the side with the concave shape which has a convex shape.

14. The method of claim 13, wherein the concave side and the convex side each have a center-point that is substantially the same.

15. The method of claim 11, wherein the film layer has an arcuate parentheses-type shape allowing the film layer to be positionable adjacent to the rectum.

16. The method of claim 15, wherein the film layer of one skin barrier film is positionable on one cheek of the patient's bottom and another film layer is positionable on another cheek of the patient's bottom with the application looking like an opening and a closing parentheses similar to ( ).

17. A skin barrier film system, comprising:
a film layer being shaped to accommodate a physical attribute of a patient;
an adhesive applied to a portion of a perimeter of the film layer; and
a removable layer removably coupled to the adhesive on the film layer.

18. The skin barrier film system of claim 17, wherein the physical attribute of the patient for which the film layer is shaped is a nipple of a breast of the patient, the film layer being shaped to allow positioning of the film layer over the nipple without adhering to the skin of the nipple of the patient.

19. The skin barrier film system of claim 18, wherein the film layer has a channel without adhesive, the channel extending from a periphery of the film layer to the center of the film.

20. The skin barrier film system of claim 19, wherein the channel is arrangeable to be oriented downward so that drainage of any fluid can occur.
